(19) United States
(12) Patent Application Publication
(10) Pub. No.: US 2010/0144704 A1
(43) Pub. Date: Jun. 10, 2010

(54) Method of reducing amyloid-beta peptide levels using a bisdioxopiperazine

(76) Inventors: Nigel H. Greig, Phoenix, MD (US); Kurt Hellmann, East Sussex (GB)

Correspondence Address: Woodcock Washburn LLP, Cira Centre, 12th Floor, 2929 Arch Street, Philadelphia, PA, 19104, US

(21) Appl. No.: 12/308,027

(22) PCT Filed: Jun. 8, 2007
(86) PCT No.: PCT/US2007/013607

§ 371 Date: Feb. 24, 2010

Related U.S. Application Data
(60) Provisional application No. 60/811,836, filed on Jun. 8, 2006.

(51) Int. Cl. A61K 31/55; A61K 31/497

(52) U.S. Cl. 514/215; 514/252.11

(57) ABSTRACT

Disclosed are methods of reducing amyloid-β peptide levels in a subject. The method involves administering to the subject a therapeutically effective amount of a bisdioxopiperazine or a pharmaceutically acceptable salt thereof to reduce β-amyloid peptide levels.

### CROSS REFERENCE TO RELATED APPLICATIONS

This application claims the benefit of priority to U.S. Provisional Patent Application 60/811,836, filed Jun. 8, 2006, which is incorporated by reference herein in its entirety.

### FIELD

The disclosure relates to medicine generally, and more specifically to a method and associated materials for reducing undesirable protein aggregation, abnormal protein folding and/or hyperphosphorylation, such as reducing aggregation of β-amyloid, α-synuclein, and/or tau, using a piperazine compound, such as razoxane.

### BACKGROUND

Alzheimer disease (AD) is equated with an impaired memory, but includes a number of other changes in brain function that result in inattention, disoriented behavior, altered personality, difficulty speaking and comprehending, and impaired gait and movement.

AD is marked by accumulation of extracellular deposits of β-amyloid in brain regions that are important for memory and cognition (e.g., the hippocampus and cerebral cortex). β-Amyloid (Aβ) is comprised of 40 and 42 amino-acid peptides (Aβ40 and Aβ42), generated by proteolytic processing of a widely expressed cell surface protein called amyloid precursor protein (APP). Aβ is prone to concentration-dependent oligomerization and aggregation. Rising levels of Aβ in the brain extracellular fluid and cerebrospinal fluid gradually leads to formation of small oligomers followed by growth into protofibrils and fibrils, consequent to changes in the tertiary structure of the protein. Such oligomers/protofibrils/fibrils play a role in inhibiting LTP (Walsh et al., (2002) Naturally secreted oligomers of amyloid beta protein potently inhibit hippocampal long-term potentiation in vivo. Nature (Lond) 416: 535-9; LaFerla and Oddo (2005) Alzheimer's disease: Abeta, tau and synaptic dysfunction. Trends Mol Med 11: 170-6) and not only induce local structural disruption of synapses and neurite breakage but also result in cell death due to perturbed calcium homeostasis and oxidative stress (Sambamurti et al., (2002) Advances in the cellular and molecular biology of the beta-amyloid protein in Alzheimer's disease. Neuromol Med 1:1-31; Gong et al., (2003) Alzheimer's disease-affected brain: presence of oligomeric A beta ligands (ADDLs) suggests a molecular basis for reversible memory loss. Proc Natl Acad Sci USA 100: 10417-22). They can, additionally, associate with other peptides and proteins to form highly insoluble neuritic plaques. The buildup of Aβ aggregates in the AD brain may be followed by formation of intracellular neurofibrillary tangles and activation of local inflammatory reactions. These brain changes ultimately lead to a widespread loss of synapses, neuronal degeneration, and neurotransmitter deficits. The development of AD is marked by progressive decline in memory and language function, personality changes, and finally dementia. In the year 2000, there were an estimated 4.5 million people in the U.S. with AD, and this number is predicted to triple by 2050.

As explained in Cuajungco et al.; Metal Chelation as a Potential Therapy for Alzheimer's Disease, Annals New York Academy of Sciences 920:292-304 (2000), the entirety of which is incorporated by reference, the lack of effective treatments makes it imperative to find new pharmacotherapies for AD, and, reports indicate the pathogenesis of AD is linked to neocortical Aβ deposition, which is mediated by abnormal metal interaction with Aβ. It has been found that precipitation of Aβ by Zn(II) is reversible and that Zn(II)-assembled Aβ can be resolubilized by chelation. However, the ability of a chelating compound to achieve such results is difficult to predict. Various chelators can act to either deprive biological systems of metal ions or can have the opposite effect of promoting metal uptake into cells.

Further, research suggests that the ability of a compound to inhibit Aβ: metal-mediated redox activity is not simply a product of affinity, but that other factors play important roles. For example, desferrioxamine (DFO), a high affinity Fe(III) chelator was initially reported to significantly arrest progression of AD. However, these results were not reproduced and further research into DFO was met with diminished enthusiasm. Reportedly, DFO exhibited an apparent reduction of its membrane permeability when in an iron bound form, compared to the unbound form. As DFO was only able to cross the blood/brain barrier in the unbound state, administration resulted in the collection of the bound form in the brain. Thus, factors other than affinity, such as bioavailability, absorption, excretion, or toxicity can affect the ability of a compound to inhibit Aβ: metal-mediated redox activity.

Current treatment for AD includes the administration of acetylcholinesterase inhibitors to increase the available acetylcholine by blocking the degradation of this neurotransmitter by acetylcholinesterase. Acetylcholinesterase inhibitors that have been approved for use in the treatment of AD include tetrahydroaminoacridine (also known as tacrine), donepezil, galantamine (also known as galanthamine), and rivastigmine. While administration of acetylcholinesterase inhibitors may increase cognition and mental function in mildly affected Alzheimer's patients, the progression of AD is not known to be retarded thereby. Likewise, the NMDA antagonist memantine was recently approved for the treatment of AD, and while it may slow the cognitive decline, the medical community still seeks a new drug that can delay or even stop progression of the disease.

Likewise, accumulating evidence indicates that a number of disorders that were initially thought to be quite diverse actually share a common molecular basis, for example, changes in protein confirmation (e.g., abnormal protein folding) (see, Thomas et al., (1999) Trend Biochem. Sci. 20:456-9; Soto, (1999) J. Mol. Med. 77:412-8; and WO 01/34631). Examples of such diseases include Alzheimer's disease, tauopathies, synucleinopathies, and/or prion based diseases.

It is therefore evident that a need exists for additional therapies for reduction of protein aggregation in a subject, for example, reducing β-amyloid peptide levels. In addition to the reduction of β-amyloid peptide, increased deposits of α-synuclein and phosphorylated tau protein are associated with cognitive diseases that share a number of inter-relationships with Alzheimer's disease, for example, tauopathies and synucleinopathies.

### SUMMARY

In accordance with the purposes of the disclosed materials, compounds, compositions, articles, and methods, as embodied and broadly described herein, the disclosed subject matter, in one aspect, relates to compounds and compositions and methods for preparing and using such compounds and compositions. Further, the disclosed subject matter relates to medicine generally, and more specifically to a method and associated materials for reducing aggregation of β-amyloid or α-synuclein peptide levels using a bisdioxopiperazine. The disclosed subject matter also relates to a method and associated materials for reducing hyperphosphorylation of Tau using a bisdioxopiperazine. The disclosed subject also relates to a method and associated materials for reducing miss-folded protein levels in a subject. The disclosed subject matter includes methods of reducing β-amyloid and/or APP peptide levels in a subject. The disclosed methods involve administering to the subject a therapeutically effective amount of a bisdioxopiperazine or a pharmaceutically acceptable salt thereof to provide a reduction in miss-folded protein levels, including, but not limited to, β-amyloid, α-synuclein, and/or hyperphosphorylated tau peptide levels or a reduction in the accumulation thereof. In one example, the bisdioxopiperazine can be administered once a day. The administration can occur orally or by the peritoneal route.

The disclosed subject matter also includes a process for manufacturing a pharmaceutical composition for the reduction of miss-folded protein levels, including, but not limited to, β-amyloid, α-synuclein, and/or hyperphosphorylated tau peptide levels, the process including incorporating a bisdioxopiperazine into a pharmaceutical dosage form. The disclosed subject matter also encompasses pharmaceutical compositions prepared for storage or administration which comprise a therapeutically effective amount of a bisdioxopiperazine in a pharmaceutically acceptable carrier, excipient, and/or diluent.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE FIGURES

The accompanying figures, which are incorporated in and constitute a part of this specification, illustrate several aspects described below.

FIG. 1 depicts the effect of 21 day treatment with razoxane on brain levels of Aβ₁₋₄₀ peptide in male 5 month old transgenic (APP Swedish mutation+PS1 mutation) mice (means±S.D.). Razoxane 30 mg/kg once daily×21 days lowered Aβ₁₋₄₀ by 46%. Means±SEM are shown together with individual data values from each mouse brain (N=4 to 6 mice/group). Both razoxane doses were well tolerated and unassociated with toxicity (as assessed by appearance and weight).

FIG. 2 depicts the effect of 21 day treatment with razoxane on brain levels of Aβ₁₋₄₂ peptide in male 5 month old transgenic (APP Swedish mutation+PS1 mutation) mice (means±S.D.). Razoxane 30 mg/kg once daily×21 days lowered Aβ₁₋₄₂ by 29%. Means±SEM are shown together with individual date values from each mouse brain (N=4 to 6 mice/group).

FIG. 3 depicts cellular toxicity of razoxane and clioquinol against SHSY-5Y human neuronal cells (±SEM, N=4). Clioquinol is a classical chelating agent that has been shown in a small early clinical trail to have some affect on Alzheimer's disease subjects (Ritchie et al., (2003) Metal-protein attenuation with iodochlorhydroxyquin (clioquinol) targeting A amyloid deposition and toxicity in Alzheimer's disease: a pilot phase 2 clinical trial. Arch Neurol. 60:1685-91; Ibach et al., (2005) Clioquinol treatment in familiar early onset of Alzheimer's disease: a case report. Pharmacopsychiatry 38(4): 178-9; Ritchie et al., (2006) Clioquinol treatment in familiar early onset of Alzheimer's disease. Pharmacopsychiatry 39(2):80-1). At concentrations up to 100 μM, razoxane (unlike clioquinol) is well tolerated and without toxicity. (Significantly different from control: Dunnett's test, p<0.05*<0.01**.)

FIG. 4 depicts the effect of razoxane at lowering APP action in SHSY-5Y human neuronal cell culture (without toxicity—assessed separately and additionally confirmed in FIG. 3) (±SEM, N=4). Clioquinol is without affect. (Significantly different from control: Dunnett's test, p<0.05*.) Percent control levels: razoxane 81% 0.5 μM, 60% 2 μM, 53% 10 μM. Clioquinol 108% 0.5 μM, 116% 2 μM, 82% 10 μM (the latter 18% decline found with clioquinol was associated with a loss of cell viability).

FIG. 5 depicts the effect of both razoxane and dexrazoxane at lowering APP levels in SHSY-5Y human neuronal cells (±SEM, N=4). Razoxane appears to be more effective (0.5 and 10 μM: 75% and 42% control levels) than dexrazoxane (0.5 and 10 μM: 87% and 59%) in this study. Both razoxane and dexrazoxane were superior to controls. (Significantly different from control: Dunnett's test, p<0.05*.)

### DETAILED DESCRIPTION

The materials, compounds, compositions, articles, and methods described herein can be understood more readily by reference to the following detailed description of specific aspects of the disclosed subject matter and the Examples included therein and to the Figures.

Before the present materials, compounds, compositions, articles, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an agent” includes mixtures of two or more such agents, reference to “the composition” includes mixtures of two or more such compositions, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that these data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, “treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

As used herein “peptide,” “polypeptide” and “protein” include polymers of two or more amino acids of any length, and includes post-translational-modification, without restriction on length. No distinction, based on length, is intended between a peptide, a polypeptide or a protein. Further, peptide, polypeptide or protein includes fragments, such as Aβ₁₋₄₂ of the amyloid precursor protein.

As used herein “hyperphosphorylated tau” means a heterogeneous or consistent phosphorylation difference between a disease state and the wild-type (e.g., Gibb et al., (2004) Differential involvement and heterogeneous phosphorylation of tau isoforms in progressive supranuclear palsy. Brain Res Mol Brain Res. 121:95-101; and Morris et al. (2002) Pathological, clinical and genetic heterogeneity in progressive supranuclear palsy. Brain 125:969-75). As will be recognized by a person of ordinary skill in the art, phosphorylation of tau occurs in healthy subjects, however, in Alzheimer's disease and other disorders (tauopathies), tau proteins gain unusually high levels of phosphorylation, can be subject to cleavage, and generally lose the ability to function normally, which is typically evidenced by aggregation of the protein. Hence, as used herein “hyperphosphorylated tau” includes unusually high levels of phosphorylation, cleavage, loss of function, and/or aggregation of the protein. Likewise, as used herein “aggregation of tau,” or similar phrases, means hyperphosphorylated tau protein.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

Reference will now be made in detail to specific aspects of the disclosed materials, compounds, compositions, articles, and methods, examples of which are illustrated in the accompanying Examples and Figures.

The disclosure relates to medicine generally, and more specifically to a method and associated materials for reducing β-amyloid peptide levels using a bisdioxopiperazine. Razoxane (RAZOXIN™ or ICRF 159) is the racemic dl form of 1,2-bis(3,5-dioxopiperazin-1-yl)-propane, and dexrazoxane is the more soluble S(+)-enantiomer. The bis-dioxopiperazine family, to which razoxane belongs, are known, particularly as anti-tumor agents. This class of compounds, their use and preparation are described in U.S. Pat. Nos. 4,275,063 and 3,941,790, the contents of both of which are incorporated by this reference. Razoxane is commercially available. As described in Braybrooke et al., supra, razoxane is absorbed from the gastrointestinal tract in a schedule-dependent matter. The plasma half-life of razoxane in humans is about 3.5 hours. Absorption is poor with large single doses, but is satisfactory with small divided doses. In one embodiment, 125 mg is administered orally to the subject, twice a day. In another embodiment, 175 mg is administered to the subject on a daily basis. Razoxane is relatively well tolerated at an oral dose of 125 mg, twice a day for five days each week for up to two years. Razoxane has been shown to cross the blood/brain barrier.

The bisdioxopiperazine family, including razoxane, includes a number of compounds that can be useful for reducing (β-amyloid peptide levels. These compounds share the general formula:

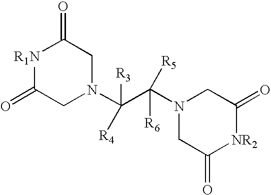

wherein R₁ can be H, CH₃ or CH₂OH; R₂ can be H, CH₃ or CH₂OH; R₃ can be H, CH₃ or C₂H₅; R₄ can be H, CH₃ or C₂H₅; R₅ can be H or CH₃; and R₆ can be H or CH₃. The compounds disclosed herein thus include (+)-1,2-Bis(3,5-dioxopiperazin-1-yl)propane and (−)-1,2-Bis(3,5-dioxopiperazin-1-yl)propane (and racemic mixtures thereof), 1,2-Bis(3,5-dioxo-1-piperazinyl)ethane, 1,2-Bis(3,5-dioxo-4-methylpiperazin-1-yl)ethane, Meso-2,3-Bis(3,5-dioxopiperazin-1-yl)butane, 1,2-Bis(3,5-dioxo-4-hydroxymethylpiperazin-1-yl)ethane, (±)-1,2-Bis(3,5-dioxopiperazinyl)butane, and (±)-1,2-Bis(3,5-dioxopiperazinyl)-2-methylpropane, in addition to other compounds satisfying this general formula.

Bisdioxopiperazines, such as razoxane (CAS Registry Number: 21416875) and dexrazoxane (CAS Registry Number: 24584096) are chelating agents and antimitotic agents with anti-inflammatory and immunosuppressive properties. Intravenous administration of dexrazoxane provides cardioprotection against anthracyline toxicity, and appears to inhibit formation of a toxic iron-anthracyline complex. The mechanism by which this cardioprotective activity occurs is not fully understood.

Razoxane, which is orally bioavailable, is an antiangiogenic topoisomerase II inhibitor that has been shown to inhibit the metastatic spread of Lewis lung 3LL, hamster lymphoma ML, and murine squamous carcinoma G cells in experimental animals and has also been shown to cause a marked increase in the sensitivity of tumors to radiation. (See e.g., Braybrooke et al., (2000) A Phase II study of razoxane, an antiangiogenic topoisomerase II inhibitor, in renal cell cancer with assessment of potential surrogate markers of angiogenesis. Clinical Cancer Research, 6:4697-704.)

Bisdioxopiperazines can be formulated for use as a pharmaceutical by a variety of methods. For instance, razoxane can be applied as an aqueous, oily (e.g., as a suspension in isopropyl myristate), or in some cases emulsified composition. Razoxane has relatively low aqueous solubility and is therefore usually (when a liquid form is desired) administered in the form of aqueous suspensions containing suitable surface active agents. It can also be administered in a tablet, capsule, or similar oral dosage form.

As will be understood by a person of ordinary skill in the art, using the guidance herein, a “therapeutically effective amount” of a compound disclosed herein will depend on the route of administration, the type of subject being treated (e.g., a human, dog, cat, horse or other warm-blooded subject), and the physical characteristics of the specific subject under consideration. These factors and their relationship to determining this amount are well known to skilled practitioners in the medical arts. This amount and the method of administration can be tailored to achieve optimal efficacy, but will depend on art recognized factors such as weight, diet, concurrent medication and other factors.

The normal daily dosage of a suitable bisdioxopiperazine lies in the range from about 10 mg to about 3 grams, from about 25 mg to about 3 grams, from about 50 mg to about 500 mg, form about 100 mg to about 400 mg, and/or about 125 mg to about 175 mg. For example, for use in reducing Aβ peptide levels, subject dosages of razoxane of from between about 10 mg to about 200 mg, or from about 10 mg/kg to about 35 mg/kg can be used. It will be appreciated that these daily dosages can be divided into two or more portions, for example three or even five, and the administration during the day of several smaller doses can prove advantageous as compared with a single larger dose. Furthermore, the daily dosage will vary somewhat according to the particular subject and the mode of administration. Thus, doses as indicated herein can be given as a solution for intravenous injection by slow infusion, by the intramuscular route, or in small volumes subcutaneously. In some instances, however, and particularly in the case of oral administration, the daily dosage can be selected in a range with a higher minimum and maximum, for example from 25 or 500 milligrams up to 1 to 3 grams.

Dosage schedules are well-known in the art and include administration of a bisdioxopiperazine for a fraction of the days in a week or month, e.g., five days a week, or another fractional administration dosage schedule.

Pharmaceutically acceptable carriers, excipients, and diluents for therapeutic use are well known in the pharmaceutical art, and are described, for example, in Remington: The Science and Practice of Pharmacy (19^{th} ed.) Gennaro, ed., Mack Publishing Company, Easton, Pa., 1995, which is incorporated by reference herein for its teachings of carriers and pharmaceutical formulations. For example, sterile saline and phosphate-buffered saline at physiological pH can be used. Conventional carrier materials such as starch, lactose, dextrin, and magnesium stearate can also be used in the pharmaceutical compositions. Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The pharmaceutical compositions can be formulated for oral administration, e.g., pills, tablets, or capsules, as well as other types of formulations including, aerosols, cachets, suppositories, etc.

When a bisdioxopiperazine is to be formulated as a pharmaceutically acceptable salt, preferred formulations can be prepared with methane sulphonic acid, isethionic acid, tartaric acid and other solubilizing acids. Salts thus formed are frequently difficult to isolate in view of the weak basicity of some of the parent compounds but their aqueous solutions, after adjustment to physiologically acceptable pH with buffers, are typically stable for extended periods of time. Solutions of similar strength, i.e., 0.5% (w/v), are also obtainable with hydrochloric acid. The mesylate salt can also be used.

Pharmaceutically acceptable salts include tartrate, formate, citrate, salicylate, fumerate, oxalate, phosphate, succinate, maleate, phenylsuccinate, hydrochloride, hydrobromide, sulfonate, benzenesulfonate, naphthalenesulfonate, hydroidate, sulfamate, sulfate, acetate, trifluoroacetate, trichloroacetate, gluconate, benzoate, lactate, methanesulfonate, ethanesulfonate, benzenesulfonate, choline hydrochlorate, p-toluenesulfonate, cyclolexylsulfonate, cyclohexylsulfamate, quinate, bicarbonate, bisulfate, bitartrate, borate, bromide, calcium edetate, camsylate, carbonate, chloride, clavulanate, dihydrochloride, edetate, edisylate, estolate, esylate, gluceptate, gluconate, glutamate, glycollylarsanilate, hexylresorcinate, hydrabamine, hydroxynaphthoate, iodide, isethionate, lactobionate, laurate, malate, mandelate, mesylate, methylbromide, methylnitrate, methylsulfate, monopotassium maleate, mucate, nitrate, N-methylglucamine, glucoheptonate, laurylsulphonate, pamoate (embonate), palmitate, pantothenate, diphosphate, polygalacturonate, potassium, sodium, stearate, subacetate, tannate, teoclate, triethiodide, trimethylammonium, oleate and/or valerate.

Reducing aggregation and/or abnormal protein folding, e.g., reducing β-amyloid and/or amyloid precursor protein levels, in a subject (e.g., a warm-blooded mammal, such as a dog, cat, horse, or human) can be beneficial in treating the symptomatic effects and/or delaying disease progression in a number of cognitive or neurological diseases or conditions, including, but not limited to, Alzheimer's disease, corticobasal degeneration, progressive supranuclear palsy, Parkinsonism-dementia complex of Guam, postencephalitic parkinsonism, dementia pugilistica, pantothenate kinase-associated neurodegeneration, Pick's disease, Down's syndrome, Argyrophilic grain disease, Myotonic dystrophy, Familial British dementia, Familial Danish dementia, Gerstmann-Straussler-Scheinker syndrome, Niemann-Pick type C disease, sclerosing panencephalitis, Parkinson's disease, prion based diseases, such as spongiform encephalopathies, Scrapie, transmissible mink encephalopathy, chronic wasting disease, bovine spongiform encephalopathy, Creutzfeld-Jacob disease, variant Creutzfeldt-Jakob disease, Gerstmann-Straussler-Scheinker syndrome, Fatal familial Insomnia, Kuru, and Alpers Syndrome, and dementia with Lewy bodies (see, Galpern and Lang, (2006) Interface between Tauopathies and Synucleinopathies: A Tale of Two Proteins. Ann. Neurol. 9:449-58; Lee et al., (2004) More than just two peas in a pod: Common amyloidogenic properties of tau and α-synuclein in neurodegenerative diseases. Trends Neurosci. 27(3):129-34; and Sigurdsson et al., (2003) Copper chelation delays the onset of prion disease. J. Biol. Chem. 278(47):46199-202, of which the entirety of each is incorporated by reference).

One of the fundamental markers for tauopathies is the increased phosphorylation of the tau protein, for example, at S396, S404 and/or S202, which can be identified by techniques well known in the art, e.g., immunoblotting and immunocytochemical studies using antibodies such as PHF-1 and AT8. See also, Spillantini et al. (1997), Familial multiple system tauopathy with presenile dementia: A disease with abundant neuronal and glial tau filaments, Proc Natl Acad Sci USA. 94(8):4113-8.

Prion proteins and prion based diseases can be assayed using any of the techniques known in the art, for example, identification of the protease resistant prion protein using immunoblotting, and measurement of the ratio of α-helices and β-sheets.

Additionally, in subjects with Down's Syndrome, there is a 1.5-fold elevation in APP levels due to three copies of chromosome 21 on which APP is expressed. These increased levels of APP can leads to elevated production and deposition of Aβ. As such, disclosed herein are methods of reducing Aβ levels in a subject with Down's Syndrome by administering to said subject a therapeutically effective amount of a bisdioxopiperazine or a pharmaceutically acceptable salt thereof, as disclosed herein.

Treatment with additional therapeutic agents, such as an acetylcholinesterase inhibitor (e.g., tetrahydroaminoacridine, donepezil, galantamine, and rivastigmine), memantine, levodopa, a dopamine agonist (e.g., bromocriptine, pergolide, pramipexole, and ropinirole), a COMT inhibitor (e.g., entacapone and tolcapone), a monoamine oxidase inhibitor (e.g., selegiline), and/or a serotonin reuptake inhibitor, can continue while the subject is undergoing bisdioxopiperazine therapy. These additional therapeutic agents can be co-administered with the bisdioxopiperazine agent, wherein the phrases “coadministration,” “in combination with,” “the combination of” or similar phrases referring to two or more drugs or compounds, means that the compounds are present in the subject being treated at the same time so as to provide the desired enhancement of treatment effect. Suitable dosing intervals and the order of administration will be readily apparent to those skilled in the art, in light of the present disclosure.

The disclosed subject matter is further explained by way of the following illustrative examples.

### EXAMPLES

The following examples are set forth below to illustrate the methods and results according to the disclosed subject matter. These examples are not intended to be inclusive of all aspects of the subject matter disclosed herein, but rather to illustrate representative compositions, methods, and results. These examples are not intended to exclude equivalents and variations of the present invention which are apparent to one skilled in the art. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example I

Double transgenic male mice (APP Swedish mutation+PS1 mutation) express human amyloid precursor protein (APP) and amyloid-β peptide (Aβ) within their brain and form amyloid depositions from 4 months onward. Razoxane (ICRF 159) was administered by intraperitoneal (i.p.) injection once daily for 21 consecutive days to a group of such mice, 5 to 6 months of age and approximately 22 to 28 g in weight. Control animals (similar littermates) received vehicle by the same route. Within three hours of the final razoxane/vehicle administration, animals were killed, their brain exposed and a 50-80 mg sample of cerebral cortex collected and frozen at ±80° C. Brain samples were then probed for human Aβ by a specific sandwich ELISA utilizing a similar capture antibody and sensitive and specific detection antibodies to both Aβ₁₋₄₀ and Aβ₁₋₄₂.

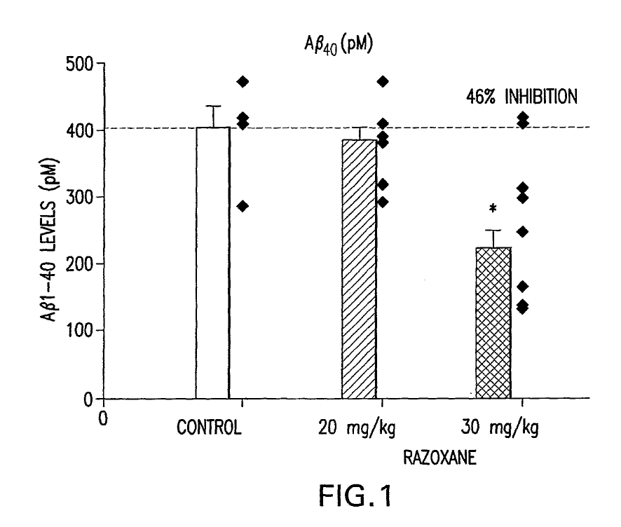

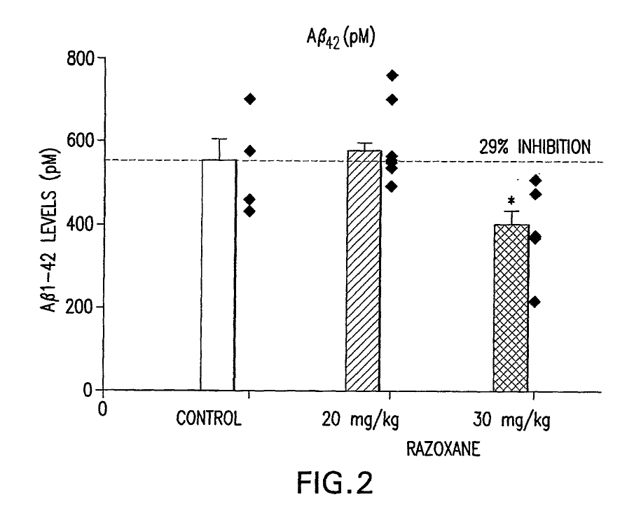

As depicted in FIGS. 1 and 2, Aβ₁₋₄₀ and Aβ₁₋₄₂ were reduced by 46% (p=0.008) 29% (p=0.088), respectively, in the razoxane treated group compared to the control group. Primary data for each group is depicted in Table I, with concentrations expressed in pM.

TABLE I

| | | | | | |
|---|---|---|---|---|---|
| Ctrl | OD | Conc | Ctrl | OD | Conc |

| | |
|---|---|
| Aβ₁₋₄₀ | Aβ₁₋₄₂ |

| | | | | | |
|---|---|---|---|---|---|
| D4 | 3.125 | 406.987 | D4 | 2.52 | 459 |
| E4 | 3.233 | 415.637 | E4 | 3.17 | 574 |
| F4 | 1.782 | 284.593 | F4 | 2.35 | 430 |
| G4 | 3.923 | 468.154 | G4 | 3.89 | 701 |
| | Average | 394 | | Average | 541 |
| | SD | 78 | | SD | 123 |

| | |
|---|---|
| Razoxane | Razoxane |
| 20 mg/kg | 20 mg/kg |

| | | | | | |
|---|---|---|---|---|---|
| B6 | 2.099 | 316.435 | B6 | 2.69 | 489 |
| C6 | 2.88 | 386.757 | C6 | 3.01 | 545 |
| D6 | 2.787 | 378.84 | D6 | 2.94 | 533 |
| E6 | 3.12 | 406.557 | E6 | 3.1 | 562 |
| F6 | 1.837 | 290.284 | F6 | 3.05 | 553 |
| G6 | 3.923 | 468.154 | G6 | 3.89 | 701 |
| | Average | 375 | | Average | 564 |
| | SD | 64 | | SD | 72 |

| | |
|---|---|
| Razoxane | Razoxane |
| 30 mg/kg | 30 mg/kg |

| | | | | | |
|---|---|---|---|---|---|
| B8 | 0.781 | 162.992 | B8 | 1.98 | 365 |
| C8 | 2.035 | 310.189 | C8 | 2.77 | 504 |
| D8 | 1.888 | 295.464 | D8 | 2.59 | 471 |
| E8 | 0.563 | 129.452 | E8 | 1.13 | 216 |
| F8 | 0.596 | 134.783 | F8 | 0.06 | 27.2 |
| G8 | 1.415 | 244.537 | G8 | 2.01 | 370 |
| | Average | 213 | | Average | 326 |
| | SD | 81 | | SD | 178 |

### Example II

Pharmaceutical dosage forms for oral administration are made by incorporating, for example, 125 mg or 175 mg of razoxane into capsules or tablets.

### Example III

The 125 mg pharmaceutical dosage forms of EXAMPLE II are administered on a twice daily basis to subjects diagnosed as having elevated amyloid-β peptide levels. Some of the subjects are already receiving acetylcholinesterase therapy, and continue that therapy.

### Example IV

The 175 mg pharmaceutical dosage forms of EXAMPLE II are administered on a daily basis to subjects diagnosed as having elevated amyloid-β peptide levels. Some of the subjects are already undergoing acetylcholinesterase therapy, and continue with that therapy.

### Example V

Pharmaceutical dosage forms for oral administration are made by incorporating, for example, 125 mg or 175 mg of dexrazoxane into capsules or tablets.

### Example VI

The pharmacokinetics of razoxane compared to those of dexrazoxane. Dexrazoxane appears to have better pharmacokinetic parameters than razoxane, as it is eliminated more rapidly from the body then razoxane. This can be more advantageous for accelerated reduction of Aβ peptide levels during initial treatments. Razoxane is observed to have a slower elimination as a consequence of slower release from the gut. This slow release property can be particularly useful for maintenance of lowered Aβ peptide levels.

### Example VII

### Assessment of Drugs in Cellular Models of Alzheimer's and Parkinson's Diseases

Parkinson's disease (PD) is characterized by the polymerization of wild-type (WT) or mutant alpha-synuclein (AS) into aggregates and fibrils. These are observed as Lewy bodies (LBs) and Lewy neurites (LNs) in PD patients and, additionally, are found in other neurodegenerative diseases, such as Lewy body dementia (LBD) and Alzheimer's disease (AD). Whereas AS is produced by a number of cell types in culture and can be quantified by Western blot analysis, its aggregation in vitro is rare, but can be induced following the transfection of human neuroblastoma SH-SY5Y (5Y) cells with mutant AS constructs (Panday et al., (2006) Exp. Neurol., 197:515-20). A variety of constructs have been described, including A30P, A53T, E46K and a glycine deletion mutation, E46KΔG. Western blot analysis demonstrated the formation of high molecular weight AS aggregates in up to 40% of cells, compared to WT, in the E46KΔG construct, which provides an assay for aggregate lowering drugs. Microscopic analysis has demonstrated inclusions that are reminiscent of those observed in PD postmortem brain samples. Hence, the assay provides a method of screening agents that lower AS and/or its aggregation that can have therapeutic value in PD. Western blot analysis of cells having either WT AS or a mutant AS construct have been used to quantify AS levels by measurement of the 14 and >188 kda protein bands.

In addition, 5Y cells (WT) incubated with drugs can be probed for relative changes in markers of Alzheimer's disease (Aβ) and tauopathies (phospho-tau).

### Example VIII

Constructs: To study the phenomenon of aggregation, a specific AS mutants (E46KΔG) is generated through polymerase chain reaction (PCR) and cloned into a pcDNA 3.1 (Invitrogen) vector. Alternatively, a GFP fusion protein of the construct is generated by in-frame cloning of AS constructs in EGFPN-1 (Clontech) vector. The sequence of the mutant is confirmed by the Sanger's Dideoxy-mediated chain termination method.

Cell culture and immunofluorescence: 5Y cells are grown in Iscove's modified Dulbecco's medium (IMDM, Invitrogen, USA), supplemented with 10% fetal calf serum (Invitrogen, USA) and 100 U/ml penicillin, 100 μg/mL streptomycin at 37° C. in 5% CO2. Cells are transfected by Lipofectamine-2000 (Invitrogen, USA) according to the manufacturer's protocol. In an exemplary embodiment, one day prior to transfection, cells are trypsinized and plated at 1×106 to 3×106 cells per well in a 6-well plate (Nunc, USA). A 5 μg sample of an appropriate plasmid (which can be estimated by O.D.) is diluted in 250 μL of OPTI-MEM medium (Invitrogen, USA) and mixed with an equal volume of OPTI-MEM medium containing 5 μL Lipofectamine 2000. The mix is then incubated for 30 min at room temperature and is added directly to the cells in the 6-well plate. Thereafter, the cells are allowed to grow in OPTI-MEM medium for 6 h, at which point the media is replaced with normal IMDM medium containing antibiotics. Cells are grown for a period of about 48-72 h before either harvesting them for immunoblot analysis or fixing them for immunofluorescence assays.

In an exemplary embodiment, AS aggregates are counted in duplicate using 100 transfected cells. Aggregates can be counted at ×20 magnification, and confirmed under higher magnifications, e.g., ×40 and ×60.

Immunofluorescence: Immunofluorescence can be performed in accord with protocols by Jana et al., (2000) Polyglutamine length-dependent interaction of Hsp40 and Hsp70 family chaperones with truncated N-terminal huntingtin: their role in suppression of aggregation and cellular toxicity. Hum. Mol. Genet. 9:2009-18. The AS antibody, syn202, can be utilized at a dilution of 1:2000 (Tu et al., (1998) Glial cytoplasmic inclusions in white matter oligodendrocytes of multiple system atrophy brains contain insoluble alpha-synuclein. Ann. Neurol. 44:415-22).

Western blot analysis: Harvested cells can be analyzed by sonication and lysing, with the samples being centrifuged at 12,000 G for 10 min at 4° C. A 25 μg sample of supernatant can then be electrophoresed on NuPAGE 4-12% Bis-Tris Gels (Invitrogen) and transferred to a polyvinylidene difluoride (PVDF) membrane (Millipore, Bedford, Mass.). Immunoblotting is performed according to standard protocols (Jana et al., 2000, ibid). In an exemplary embodiment, the primary antibodies are Syn202 (dilution of 1:4000) and LB509 (dilution 1:200) (Baba et al., (1998) Aggregation of alpha-synuclein in Lewy bodies of sporadic Parkinson's disease and dementia with Lewy bodies. Am. J. Pathol. 152:879-84). As an external control, α-actin is utilized to insure equal loading (Abeam, dilution 1:1000). Horseradish peroxidase-conjugated secondary antibody (Vector, dilution 1:6000) can be used for visualization, for example, using enhanced chemiluminescence (ECL) Plus (Amersham Biosciences, Piscataway, N.J.), following the manufacturer's protocol.

Aggregates: Following transfections into a 5Y cell line, the number of aggregates is quantified in cells. Previous studies utilizing either the mutant AS constructs or the AS-GFP fusion constructs have demonstrated that some 40% of E46KΔG transfected cells develop aggregates, compared to 6% in WT. These studies detected no aggregates in either mock transfections or GFP alone transfections. Western blot analysis of 4-12% Bis-Tris polyacrylamide gels, demonstrated that soluble AS migrates in accord with it molecular weight—with a band at 14 kDa. The density of this band did not correlate with the number of aggregates assessed by immunofluorescence. However, a higher molecular weight aggregate band was clearly evident at the top of each lane of the gel with a greater than 188 kDa. The intensity of this band was in proportion to aggregate number, and was present for WT. Thus, aggregation can be assayed using methods know in the art.

Drug treatment: compounds (0.1 to 100 uM) or vehicle are added after the completion of transfection or to WT cells at the time the medium is replaced with normal IMDM medium containing antibiotics. Thereafter cells are grown for a period of about 48-72 h before either harvesting them for immunoblot analysis or fixing them for immunofluorescence assays. The number of aggregates is assessed in comparison to E46KΔG transfected and WT cells.

### Example IX

### Tauopathy and Alzheimer's Disease Models: Phospho-Tau and Aβ Quantification in Human Neuroblastoma Cells

Cell culture: SH-SY5Y (5Y) neuroblastoma cells are grown in minimum essential medium (MEM) containing 10% fetal calf serum (FCS), 100 μg/mL streptomycin sulphate, 100 U/mL penicillin G and 1-glutamine (designated complete medium) at 37° C. in 5% CO2. Approximately sixteen hours prior to treatment cells are washed free of FCS-containing medium and are incubated in FCS-free MEM containing the neuroblastoma growth supplement N2 (cell culture materials are available from Gibco, Life Technologies).

Drug treatment: Vehicle and/or a drug candidate can be added at the desired concentration, including concentrations ranging from 0.1 to 100 μM, and incubated with cultured cells for an appropriate period of time, for example, from about 6 to about 48 hours. MTT assays are performed, preferably in duplicate or triplicate, to assess viability, according to the manufacture's guide.

Aβ-ELISAs: Samples are prepared as described and known in the art (see, Olivieri et al. (2000) Mercury induces cell cytotoxicity, oxidative stress and increases β-amyloid secretion, tau-phosphorylation in SHSY5Y neuroblastoma cells. J. Neurochem. 74:231-6). Aβ₁₋₄₀ and Aβ₁₋₄₂ levels can be determined by a sandwich ELISA, or other methods known in the art, (see e.g., Olivieri et al. 2000, ibid). In an exemplary embodiment, microtitre plates (Nunc) are sensitized with strepavidin (Roche Biochemicals) overnight. Primary capture antibody, e.g., biotinylated 6E10, 1 μg/mL (Senetek), specific for Aβ1-17 (Kim et al., (1990) Detection and quantitation of amyloid β-peptide with 2 monoclonal antibodies. Neurosci. Res. Common. 7:113-22) is added for about 8 h. Concentrated (50-fold) cell culture supernatants are diluted to 1 mg/mL with assay buffer (50 mM Tris-HCl pH 7.5 containing 140 mM NaCl, 5 mM EDTA, 0.05% NP40, 0.25% gelatine, and 1% BSA) and incubated for about 24 h at 4 C. Discrimination between Aβ₁₋₄₀ and Aβ₁₋₄₂ is made possible by the use of peroxidase labeled BAP-17 and BAP-15 antibodies specific for Aβ₁₋₄₀ and Aβ₁₋₄₂, respectively (Brockhaus et al., (1998) Caspase mediated cleavage is not required for the activity of presenilins in amyloidogenesis and NOTCH signaling. Neuroreport 9:1481-6). Each assay plate can include a standard curve with highly purified Aβ₁₋₄₀ and Aβ₁₋₄₂ (Dobeli et al. (1995) A biotechnological method provides access to aggregation competent monomeric Alzheimer's 1-42 residues amyloid peptide. Biotechnology (NY) 13:988-93). After color development with tetramethylbenzidine (Roche Biochemicals) the plates are analyzed using a plate reader (Labsystems). Using this methodology, no cross-reactivity between Aβ₁₋₄₀ and Aβ₁₋₄₂ is expected. However, alternative methodologies known in the art can be used and any cross-reactivity accounted for in the analysis.

Tau-ELISA: Phosphorylated tau levels can be determined using a number of techniques known in the art, including by a solid-phase, non-competitive sandwich ELISA (see, Herrmann et al., (1999) ELISA-Quantitation of phosphorylated tau-protein in the Alzheimer's disease brain. Eur. Neurology 42:205-10). In this exemplary embodiment, black microtitre plates (Black Cliniplate EB, Labsystems, Helsinki, Finland) are coated overnight with monoclonal antibodies, such as AT180 and AT270 (Innogenetics), both antibodies can be used at about 5 μg/mL. AT180 and AT270 recognize epitopes of tau containing phosphorylated Thr-231 and Thr-181, respectively (Goedert et al., (1994) Epitope mapping of monoclonal antibodies to paired helical filaments of Alzheimer's disease: identification of phosphorylation sites in tau protein. J. Biochem. 301:871-7). After washing, 100 μl, per well of cell lysate (120 μg per well) is incubated for about 3 h at room temperature (20-24° C.). After washing, horseradish-peroxidase-labeled mAbAD2 is added for a further 2 h at room temperature. Monoclonal antibody AD2 has been shown to have no cross-reactivity with normal adult brain tau and detects phosphorylated Ser-396 and Ser-404 (Buée-Scherrer et al., (1996) AD2, a phosphorylation-dependent monoclonal antibody directed against tau proteins found in Alzheimer's disease. Mol. Brain. Res. 39:79-88). Each assay plate can include a standard curve with phosphorylated human recombinant tau, with quantification determined by the detection of chemiluminescence. Tau-ELISAs are preferably performed in duplicate or triplicate.

Phospho-tau Western blot analysis: In another exemplary embodiment, altered tau phosphorylation, which may or may not be measurable via ELISA, 5Y cell lysates (e.g., 30 μg total protein per lane) are run on a 7.5% polyacrylamide SDS gel, blotted onto nitrocellulose, blocked overnight and probed with appropriate antibodies, such as AT180, AT270 and AD2. Bands on the blots can be visualized and/or quantified through a chemiluminescent reaction involving the addition of specific peroxidase-labelled secondary antibodies (Roche Biochemicals) and a substrate (SuperSignal, Pierce, USA).

### Example X

Cell Culture. The human neuroblastoma cell line, SHSY-5Y, was obtained from the American Type Culture Collection, culture medium was from Mediatech, Inc. (Herndon, Va.) and FCS from HyClone (Logan, Utah).

Drug Treatment. SHSY-5Y cells were cultured on 60 or 100 mm dishes at a concentration of 3 or 8×10⁶ cells, respectively. Cells were allowed to grow in complete media (10% FCS, 2 mM glutamine in DMEM) for 2 days to reach 70% confluence. Thereafter, spent media was removed and replaced with fresh media (2 mL DMEM) containing 0 to 100 μM of freshly prepared compounds (clioquinol, razoxane and dexrazoxane. Cells were incubated at 37° C., 5% CO₂ for 16 h.

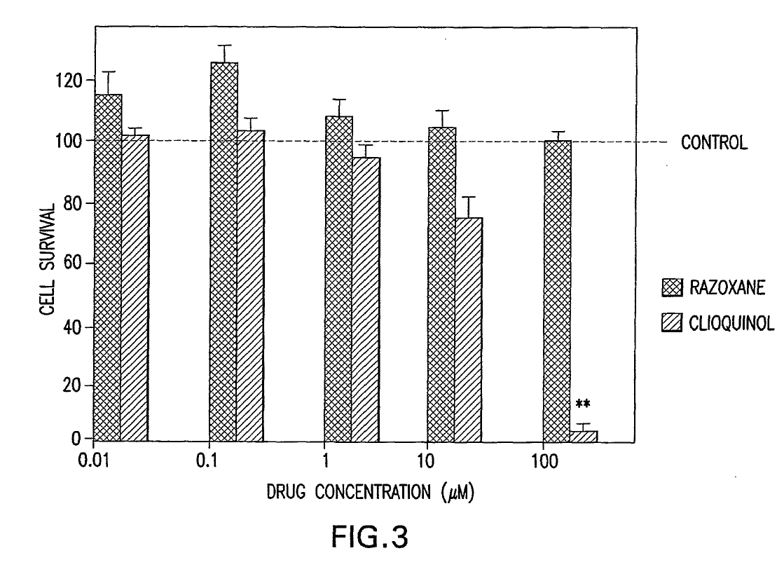

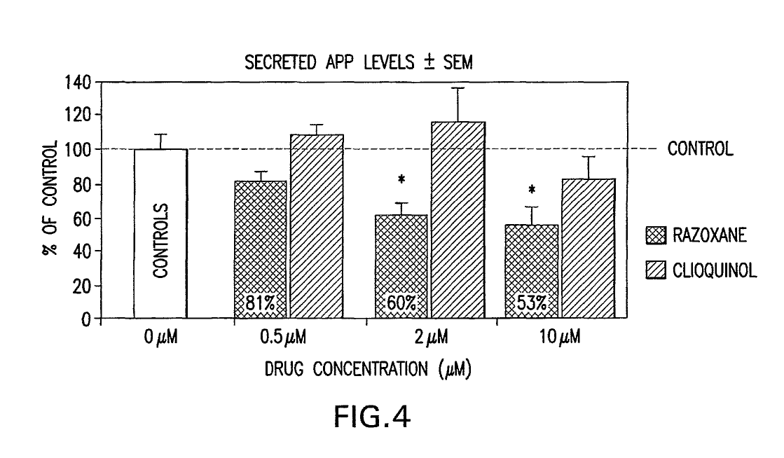

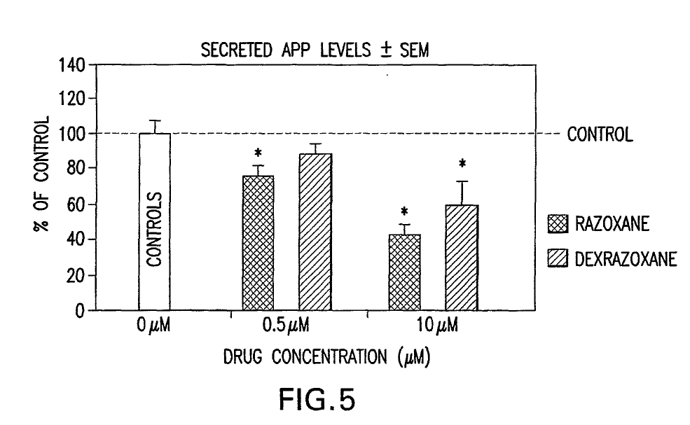

APP Western Blot and cell viability. Fifteen μg of protein from each sample was mixed with Laemmli buffer, boiled for 5 min at 100° C., and loaded onto a 10% SDS PAGE gel (Novex, San Diego, Calif.). The proteins separated at 150V for 90 min, transferred to nitrocellulose membrane and probed with 22C11 (2 μg/mL) antibody to APP. The blots were incubated in secondary antibody, anti-mouse IgG—conjugated to horseradish peroxidase, for 30 min. Thereafter, samples were detected by chemiluminescence. Cell viability was assessed by a sensitive MTT assay. Values were expressed as the mean of between four and six independent assays and were statistically compared to control values. (See FIGS. 3-5).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A method of reducing amyloid-β peptide levels in a subject, said method comprising: administering to the subject a therapeutically effective amount of a bisdioxopiperazine or a pharmaceutically acceptable salt thereof, wherein the bisdioxopiperazine has the general formula 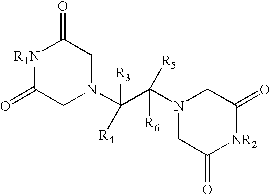
wherein R₁ is H, CH₃ or CH₂OH,
R₂ is H, CH₃ or CH₂OH,
R₃ is H, CH₃ or C₂H₅,
R₄ is H, CH₃ or C₂H₅,
R₅ is H or CH₃, and
R₆ is H or CH₃; and
reducing amyloid-β levels in the subject.

2. The method according to claim 1, wherein administering to the subject a therapeutically effective amount of a bisdioxopiperazine or a pharmaceutically acceptable salt thereof comprises administering to the subject a therapeutically effective amount of razoxane, dexrazoxane, or a pharmaceutically acceptable salt thereof.

3. The method according to claim 2, comprising orally administering a therapeutically effective amount of razoxane, dexrazoxane, or a pharmaceutically acceptable salt thereof to the subject.

4. The method according to claim 3, comprising orally administering razoxane in an amount of about 100 mg to about 200 mg each day.

5. The method according to claim 3, wherein the razoxane, dexrazoxane, or a pharmaceutically acceptable salt thereof is administered for a period of up to about two years.

6. The method according to claim 1, further comprising: co-administering to the subject a therapeutically effective amount of an acetylcholinesterase inhibitor or NMDA antagonist.

7. The method according to claim 6, comprising co-administering the NMDA antagonist memantine.

8. The method according to claim 6, comprising co-administering the acetylcholinesterase inhibitor donepezil, galantamine, or rivastigmine.

9. The method according to claim 2, comprising intraperitoneally administering a therapeutically effective amount of razoxane, dexrazoxane or a pharmaceutically acceptable salt thereof to the subject.

10. The method according to claim 9, in an amount of from about 100 mg to about 200 mg each day.

11. The method according to claim 9, wherein razoxane is administered for a period of up to about two years.

12. The method according to claim 9, further comprising: co-administering to the subject a therapeutically effective amount of an acetylcholinesterase inhibitor.

13. The method according to claim 12, wherein administering to the subject a therapeutically effective amount of an acetylcholinesterase inhibitor comprises administering to the subject a therapeutically effective amount of an acetylcholinesterase inhibitor selected from the group consisting of phenserine, donepezil, galantamine, and rivastigmine.

14. The method according to claim 1, further comprising: co-administering to the subject a therapeutically effective amount of an agent selected from the group consisting of levodopa, bromocriptine, pergolide, pramipexole, ropinirole, and selegiline.

15. The method according to claim 1, comprising manufacturing a medicament containing a bisdioxopiperazine or a pharmaceutically acceptable salt thereof and a pharmaceutically acceptable carrier, excipient, or diluent.

16. The method according to claim 1, wherein the subject has Alzheimer's disease.

17. The method according to claim 1, wherein the subject has Down's syndrome.

18. A method of reducing aggregation of alpha-synuclein or tau protein in a subject, said method comprising: administering to the subject a therapeutically effective amount of a bisdioxopiperazine or a pharmaceutically acceptable salt thereof, wherein the bisdioxopiperazine has the general formula 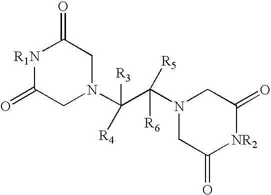
wherein R₁ is H, CH₃ or CH₂OH,
R₂ is H, CH₃ or CH₂OH,
R₃ is H, CH₃ or C₂H₅,
R₄ is H, CH₃ or C₂H₅,
R₅ is H or CH₃, and
R₆ is H or CH₃; and
reducing the aggregation of alpha-synuclein or tau protein in the subject.

19. The method according to claim 18, wherein administering to the subject a therapeutically effective amount of a bisdioxopiperazine or a pharmaceutically acceptable salt thereof comprises administering to the subject a therapeutically effective amount of razoxane, dexrazoxane, or a pharmaceutically acceptable salt thereof.

20. The method according to claim 19, comprising orally administering a therapeutically effective amount of razoxane, dexrazoxane, or a pharmaceutically acceptable salt thereof to the subject.

21. The method according to claim 20, comprising orally administering razoxane in an amount of about 100 mg to about 200 mg each day.

22. The method according to claim 20, wherein the razoxane, dexrazoxane, or a pharmaceutically acceptable salt thereof is administered for a period of up to about two years.

23. The method according to claim 18, further comprising: co-administering to the subject a therapeutically effective amount of an agent selected from the group consisting of lithium, carbidopa, levodopa, selegiline, a dopamine agonist, a COMT inhibitor, an antipsychotic, and a combination thereof.

24. The method according to claim 23, comprising co-administering levodopa or carbidopa.

25. The method according to claim 18, further comprising: co-administering an acetylcholinesterase inhibitor.

26. The method according to claim 18, comprising intraperitoneally administering a therapeutically effective amount of razoxane, dexrazoxane or a pharmaceutically acceptable salt thereof to the subject.

27. The method according to claim 18, comprising reducing aggregation of tau protein in a subject.

28. The method according to claim 18, comprising reducing aggregation of alpha-synuclein protein in a subject.

29. The method according to claim 18, comprising manufacturing a medicament containing a bisdioxopiperazine or a pharmaceutically acceptable salt thereof and a pharmaceutically acceptable carrier, excipient, or diluent.

30. A method of reducing abnormal protein folding or aggregation of abnormally folded proteins in a subject, said method comprising: administering to the subject a therapeutically effective amount of a bisdioxopiperazine or a pharmaceutically acceptable salt thereof, wherein the bisdioxopiperazine has the general formula 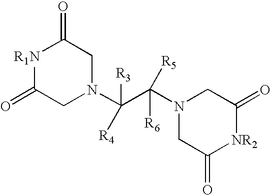
wherein R₁ is H, CH₃ or CH₂OH,
R₂ is H, CH₃ or CH₂OH,
R₃ is H, CH₃ or C₂H₅,
R₄ is H, CH₃ or C₂H₅,
R₅ is H or CH₃, and
R₆ is H or CH₃; and
reducing abnormal protein folding or aggregation of abnormally folded proteins in the subject.

31. The method according to claim 30, wherein administering to the subject a therapeutically effective amount of a bisdioxopiperazine or a pharmaceutically acceptable salt thereof comprises administering to the subject a therapeutically effective amount of razoxane, dexrazoxane, or a pharmaceutically acceptable salt thereof.

32. The method according to claim 31, comprising orally administering a therapeutically effective amount of razoxane, dexrazoxane, or a pharmaceutically acceptable salt thereof to the subject.

33. The method according to claim 30, comprising orally administering razoxane in an amount of about 100 mg to about 200 mg each day.

34. The method according to claim 30, wherein the razoxane, dexrazoxane, or a pharmaceutically acceptable salt thereof is administered for a period of up to about two years.

35. The method according to claim 30, wherein the abnormally folded protein is a prion.
